(19) United States
(12) Patent Application Publication
(10) Pub. No.: US 2008/0145840 A1
(43) Pub. Date: Jun. 19, 2008

(54) Isothermal Titration Calorimetry Assays

(75) Inventors: Ann Eakin, Waltham, MA (US); Stewart Fisher, Waltham, MA (US); Irene Karantzeni, Waltham, MA (US); Gunther Kern, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/561,320

(22) PCT Filed: Jun. 30, 2004
(86) PCT No.: PCT/GB2004/002828

§ 371 Date: Mar. 6, 2008

Related U.S. Application Data
(60) Provisional application No. 60/484,032, filed on Jun. 30, 2003.

(51) Int. Cl. C12Q 1/68; G01N 25/20; G01N 33/00; C12Q 1/26; C12Q 1/48; C12Q 1/34; C12Q 1/00

(52) U.S. Cl. 435/6; 436/147; 436/86; 436/71; 436/94; 435/25; 435/15; 435/18; 435/4

(57) ABSTRACT

A method for identifying a modulator of a biological process or a biomolecule.

### CROSS-REFERENCE TO RELATED APPLICATIONS

This application is related to U.S. Provisional Application No. 60/484,032, filed Jun. 30, 2003, which is incorporated herein by reference in its entirety.

### FIELD OF THE INVENTION

The present invention relates to methods of identifying and characterizing a modulator of a biological process or a modulator of a specific biomolecule.

### BACKGROUND

A major process in drug discovery involves the study of biochemical systems and the specific nature of the interaction of biomolecules with various ligands. Microcalorimetric techniques such as isothermal titration calorimetry (ITC) and differential scanning calorimetry (DSC) are widely used in understanding these interactions (Jelesarov & Bosshard, 1999, J. Mol. Recognit., 12:3-18; Ward & Holdgate, 2001, Prog. Med. Chem., 38:309-76; Sturtevant, 1987, Ann. Rev. Phys. Chem., 38:463-88).

ITC is a technique used primarily in measuring the equilibrium heat of binding of a ligand to a macromolecule (Leavitt & Freire, 2001, Curr. Op. Struct. Biol., 11:560-6; Ladbury & Chowdhry, 1996, Chem. Biol., 3:791-801; Doyle, 1997, Biotechnology, 8:31-35; Fisher & Singh, 1995, Methods Enzymol., 259:194-221). In recent years ITC has also been used in the determination of enzymatic activity and enzyme kinetic parameters such as the Michaelis-Menten kinetic parameters K_{M} and k_{cat} (Todd & Gormez, 2001, Anal. Biochem., 296:179-187; Cai et al., 2001, Anal. Biochem., 299:19-23).

Several studies of enzymatic activities have been reported using the detection of reaction heat for measuring kinetic parameters (Lonhienne et al., 2001, Biochim. Biophys. Acta, 1545:349-356; Quemard et al., 1995, Biochemistry, 34:8235-8241; Silberg & Vickery 2000, J. Biol. Chem., 275:7779-7786; Prodromou et al., 1999, EMBO J., 18:754-762).

The determination of the inhibition profiles of different compounds towards a target (e.g., IC₅₀ value, the concentration of compound at which 50% of the enzymatic activity is inhibited) is of particular interest in drug discovery. Determination of such quantities as IC₅₀ and Kᵢ (kinetically determined inhibition constant representing the potency of an enzyme inhibitor) are imperative in designing novel potent inhibitors that will lead to developing effective drugs. Recent experiments describe the use of DSC in determining K_{d} (equilibrium dissociation constant) values for binding of inhibitors to uridine diphosphate-N-acetylenolpyruvylglucosamine reductase (MurB) (Sarver et al., 2002, J. Biomol. Screening, 7:21-28). ITC is also used traditionally in the determination of K_{d} values but not IC₅₀ or Kᵢ values (kinetically determined inhibition parameters).

### SUMMARY OF THE INVENTION

The present invention is based, in part, on a method that can be used to monitor the activity of a biomolecule or a biological process where, as a result of the activity, heat is released or absorbed. Reaction heat rate is defined as the amount of heat released or absorbed by the reaction per unit time. The reaction heat rate can be directly correlated to the reaction product formation rate or the reaction substrate depletion rate. By monitoring changes in the reaction heat rate, changes in the product formation rate or the substrate depletion rate can be identified and overall changes in the reaction process can be determined. For example, modulators of a biomolecule, or modulators of a biological process, can be identified by determining the difference in the reaction heat rate upon the addition of a test modulator or compound. In one example, the present invention can be used in high throughput screening (HTS) by providing a reaction system whereby prior to the addition of the modulator the steady state of reaction of the test biological process sample and a control have been heat equilibrated such that any alteration in the reaction heat rate by the modulator can be monitored.

The present invention has general applicability because the methods are based upon the determination of the reaction heat rate. Also the present methods have advantages over prior art methods. For example, the methods of the present invention do not require introduction of coupling enzymes. The use of such enzymes can cause ambiguity with respect to read out because the test compounds can inhibit either or both the target and the coupling enzymes. Moreover, calorimetric methods are devoid of artifacts and interferences that are sometimes produced in spectrophotometric and fluorometric assays.

The invention includes screening methods for identifying a modulator of a biological process. In one aspect, the method includes providing a test mixture from a biological process under conditions that support biological activity; adding a test compound to the test mixture; and detecting a difference in the reaction heat rate in the presence of the compound compared to the reaction heat rate in the absence of the test compound, wherein the difference is indicative that the test compound modulates the activity of the biological process.

In another aspect, the method includes providing a test mixture from a biological process and a control (e.g., the control can be a test mixture from the same biological process); adding a test compound to the test mixture; equilibrating heat between the test mixture and the control; initiating a reaction in the test mixture and the control; and detecting a difference in the reaction heat rate between the test mixture and control, wherein the difference is indicative that the test compound modulates the activity of the biological process.

In yet another aspect, the invention includes providing a test mixture from a biological process under conditions that promote activity and a control; equilibrating heat between the test mixture and the control; adding a test compound to the test mixture; and detecting a difference in the reaction heat rate in the presence of the compound compared to the reaction heat rate in the absence of the test compound, wherein the difference is indicative that the test compound modulates the activity of the biological process.

The biological process described above can be any process, for example, the biological process can be transcription, translation, bacterial cell wall biosynthesis, cellular respiration, cofactor biosynthesis, DNA replication, glycolysis, glucogenesis, amino acid and fatty acid biosynthesis, protein degradation, or protein secretion.

The invention also includes screening methods for identifying a compound that modulates the activity of a biomolecule. In one aspect, the method includes providing a test mixture comprising a biomolecule under conditions that support the activity of the biomolecule; adding a test compound to the test mixture; and detecting a difference in the reaction heat rate in the presence of the compound compared to the reaction heat rate in the absence of the test compound, wherein the difference is indicative that the test compound modulates the activity of the biomolecule.

In another aspect, the invention includes providing a test mixture comprising a biomolecule and a control; adding a test compound to the test mixture; equilibrating heat between the test mixture and the control; initiating a reaction in the test mixture; and detecting a difference in the reaction heat rate between the test mixture and control, wherein the difference is indicative that the test compound modulates the activity of the biomolecule.

In yet another aspect, the invention includes providing (i) a test mixture comprising a biomolecule under conditions that promote the activity of the biomolecule and (ii) a control; equilibrating heat between the test mixture and the control; adding a test compound to the test mixture; and detecting a difference in the reaction heat rate in the presence of the compound compared to the reaction heat rate in the absence of the test compound, wherein the difference is indicative that the test compound modulates the activity of the biomolecule.

The biomolecule that can be used in the methods above can be selected from the group consisting of a protein such as an enzyme or a polypeptide, an oligonucleotide, a DNA or RNA polynucleotide, a carbohydrate, and a lipid.

The enzyme can be any appropriate enzyme, for example, the enzyme can be from a prokaryote such as a bacterium, a eukaryote, a virus or a fungus. The enzyme can be involved in any biological process such as cell wall biosynthesis, transmembrane signaling, translation, transcription, replication, protein secretion, or cofactor biosynthesis. In one embodiment, the enzyme is a topoisomerase. In another embodiment, the enzyme can be DNA gyrase, topoisomerase IV or topoisomerase II. In yet another embodiment, the enzyme is selected from the group consisting of oxidases/reductases, kinases, ligases, phosphatases, MurB, uridine diphosphate-N-acetylmuramatel:L-alanine ligase (MurC) and DNA ligase.

In the screening methods described above, compounds such as peptides, peptidomimetics, small molecules, or other drugs can be tested for their ability to modulate the activity of a biomolecule or biological process.

### BRIEF DESCRIPTION OF THE DRAWINGS

FIG. 1 depicts a schematic representation of a calorimetric chamber.

FIG. 2A depicts a line graph showing inhibition of E. coli GyrAB ATPase activity by novobiocin as measured by ITC. FIG. 2B shows the data fitted to an IC₅₀ curve.

FIG. 3A depicts a line graph showing E. coli GyrAB ATPase reaction progressing to completion after each addition of substrate. FIG. 3B shows the heat calibration curve obtained by the same experiment.

FIG. 4A depicts a line graph showing inhibition of E. coli MurC activity by β,γ-methyleneadenosine 5′-triphosphate as measured by ITC. FIG. 4B shows the data fitted to IC₅₀ curve.

FIG. 5 depicts a line graph showing inhibition of E. coli MurB activity by (2R)-2-{2-[3-(4-tert-butylphenoxy)phenyl]-4-oxo-1,3-thiazolidin-3-yl} hexanoic acid) (data fitted to an IC₅₀ curve).

FIG. 6A depicts a line graph showing the heat rate observed for coupled transcription/translation, in the presence (open circles) and absence of tetracycline (closed circles), upon titrating S30 extract (containing the ribosomes) in the reaction mixture (containing DNA, amino acids, and other necessary components for the reaction to occur).

FIG. 6B depicts a line graph showing the difference in the heat rate observed in the presence and in the absence of tetracycline upon titrating S30 extract in the reaction mixture.

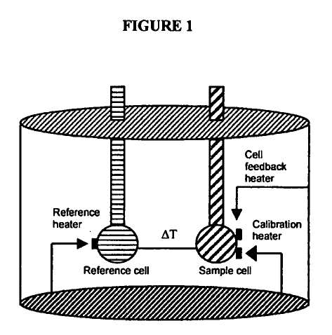

### DETAILED DESCRIPTION

The present invention provides, in part, a method that is particularly suitable for identifying modulators of a biological process. A biological process includes at least two or more biomolecules and their chemical reactants combined under conditions that promote or support activity. Suitable conditions include those conditions that allow the biomolecules involved in the biological process to react with their chemical reactants to a measurable extent. Typically, a biological process includes multiple reactions running simultaneously. By using the methods of the invention, there is no necessity to detect specific product formation or specific substrate depletion. In fact, with the methods of the present invention it is not necessary to even know the identity of any of the biomolecules or reactants within the biological process of interest. Because the methods of the present invention measure the reaction heat rate or the net change in heat resulting from multiple reactions in a biological process, the methods provide an easy and rapid means of identifying modulators of a biological process.

The invention also includes a method for identifying modulators of a particular biomolecule by monitoring changes in the reaction heat rate resulting from changes in the activity of the biomolecule in the presence and in the absence of test modulators. A biomolecule includes any chemical entity capable of catalyzing a chemical reaction in a biological process. Examples of biomolecules include, but are not limited to proteins, enzymes, polypeptides, DNA or RNA oligonucleotides, DNA or RNA polynucleotides, lipids, and carbohydrates.

The present invention also provides methods for determining inhibition profiles (such as IC₅₀ values) for compounds that inhibit a biological process or biomolecule. In some embodiments the biomolecule is an enzyme and an inhibition profile is determined for the activity of the enzyme.

Screening Methods

The methods of the present invention require measuring the reaction heat rate as a means of monitoring for changes in a chemical reaction. For example, a decreased heat rate observed in the presence of a modulator as compared to that in the absence of a modulator would indicate a decrease in the reaction rate and the modulator would be identified as an inhibitor of that particular reaction.

In one example, the present method involves providing (i) a test mixture containing a biomolecule and (ii) a control mixture against which the reaction heat rate in the test mixture will be compared (for example the control can be water; buffer; or a test mixture which does not include one of the components of the reaction). In one embodiment, the reaction in the test mixture is initiated by adding one or more substrates or activators that are necessary for the biological process. For example, in a screening method for identifying modulators of a biomolecule, the reaction in the test mixture is initiated upon adding a known substrate or activator, e.g., a cofactor or metal ion, of the test biomolecule. The next step of the method requires heat equilibration between the test and control mixtures prior to the addition of the test compound.

To achieve heat equilibration a preset differential power (DP) or baseline signal can be applied to the mixtures containing the test biomolecule and the control such that a change in temperature (ΔT) is zero or constant. Upon reaching thermal equilibrium between the test mixture and the control, a test compound is introduced into the test mixture, for example, the compound is injected into the test mixture through a titration syringe. Depending on the nature of the interaction between the biomolecule and the test compound, DP will decrease to compensate for the heat released (exothermic event) or increase to compensate for the heat absorbed (endothermic) in the test mixture while keeping ΔT between the cells zero or constant. In one example, the test compound is an inhibitor, the biomolecule is an enzyme and the reaction catalyzed is exothermic. In this example, if the enzyme is inhibited, less heat is released in the test mixture. The decrease in the heat rate (which corresponds to a decreased reaction rate due to inhibition) results in an increase in the DP signal since more power must now be provided from the feedback heater to keep the test mixture and control at the preset ΔT. The direction of the heat change would be opposite for an endothermic reaction. In another embodiment, a compound is preincubated with the test mixture and, or, the control mixture prior to heat equilibration. The control can be water; buffer; or a test mixture that does not include one of the component reactions of the test biological process.

Alternatively, the mixtures can be heat equilibrated by, for example, applying a DP or by accomplishing thermal diffusion through contact between the vessels containing the test and control mixtures. Upon reaching thermal equilibrium, the reaction can be initiated by adding a substrate or a required reaction component, for example the substrate is introduced with a multidrop cassette. The heat rate in the test mixture containing the compound will be compared to the heat rate in the test mixture lacking the compound, for example, if the reaction heat rate in the presence of compound is smaller than that in the absence of compound, the compound will be identified as a potential inhibitor of one or more of the biomolecules participating in the reaction.

In some embodiments, the biomolecule can have more than one function, for example, it can be bifunctional. Using the methods of the present invention, it is possible to determine if a test compound affects the ability of a bifunctional biomolecule to catalyze the reaction with its known substrates, referred to in this example as substrates A and B. For example, the method includes providing a test mixture which contains the bifunctional molecule with substrates A and B and providing a test mixture which contains the bifunctional molecule and only substrate A. The reactions in both test mixtures are initiated and the test compound is added to both mixtures. The reaction heat rates for the test mixtures are compared. If there is little or no difference in the reaction heat rates between the two test mixtures the compound is a modulator of the reaction between the biomolecule and substrate B. Similarly, the methods of the invention can be used to determine if the compound is a modulator of the reaction between the biomolecule and substrate A.

The methods of the invention can be used to monitor the inhibition of a biomolecule such as an enzyme. Using the method of the present invention, a test mixture containing the enzyme and a control can be equilibrated after an enzymatic reaction is started in the test mixture. The enzymatic reaction can be initiated by either adding a substrate or activator of the enzyme into the test mixture. Enzymes from any source can be monitored or modulated using the assays of the present invention, including, but not limited to, viral, bacterial, prokaryotic, eukaryotic, and cancer or disease-associated enzymes. In some embodiments, the biomolecule is a bacterial enzyme, for example, from Escherichia coli, Salmonella spp, Shigella spp, Haemophilus influenzae, Moraxella catarrhalis, Pseudomonas aeruginosa, Chlamydia spp, Legionella spp, Staphylococcus aureus, Streptococcus pneumoniae, Streptococcus pyogenes, Enterococcus faecalis, Enterococcus faecium, Staphylococcus saprophyticus, or Mycoplasma spp. The biomolecule can be a topoisomerase (such as DNA gyrase (GyrAB), or topoly). The biomolecules can be MurB, MurC, or DNA ligase. In one embodiment, the biomolecule is MurB and the chemical reactants are NADPH, flavin, and uridine diphosphate-GlcNAc-enolpyruvate (UDP-GlcNAc-EP).

In other embodiments of the present invention, the biomolecule is an enzyme selected from oxidases/reductases that utilize at least one of the following cofactors: NADPH, flavin, cobalamin, S-adensoyl methionine, ubiquinone, heme, glutathione, and/or iron sulfur clusters.

Other examples of a biomolecule include ATP or NAD+-dependent ligases, including, but not limited to, the enzymes PoIC, MurC, uridine diphosphate-N-acetylmuramoyl-L-alanyl:D-glutamate ligase (MurD), uridine diphosphate-N-acetylmuramoyl-L-alanyl-D-glutamyl meso-diaminopimelate ligase/uridine diphosphate-N-acetylmuramoyl-L-alanyl-D-glutamyl:L-lysine ligase (MurE), uridine diphosphate-N-acetylmuramoyl-L-alanyl-g-D-glutamyl-meso-diaminopimeloyl:D-alanyl-D-alanine ligase (MurF), DNA ligase; a kinase, including, but not limited to, protein serine/threonine kinases, protein tyrosine kinases; dual specificity kinases; a phosphatase, including, but not limited to, pyrophosphatase, protein phosphatases, phosphodiesterases; enzymes involved in replication including, but not limited to polymerases, DNA helicases and DNA primases, including, but not limited, to the enzymes DnaB, PolIII, DnaG, DnaE; and enzymes involved in cell wall biosynthesis including, but not limited to, MurD, MurE, MurF and 3-deoxy-D-manno-octulosonate-8-phosphate synthase (KdsA).

In some embodiments, the biomolecule is a transmembrane signaling protein, including, but not limited to, bacterial and eukaryotic two component regulatory systems, G-protein coupled receptor-G protein complexes, and JAK-STAT signaling complexes or a protein chaperone, including, but not limited to, the heat shock proteins GroEL-GroES, DnaK.

Similarly, by measuring the changes in the reaction heat rate, modulators of a biological process can be identified. The method includes providing a sample from a biological process and a control. The biological process sample is then incubated under conditions effective to permit the biomolecules of the biological process to react to a measurable extent with their chemical reactants. The biological process sample is then heat equilibrated with the control sample. Following heat equilibration, the biological sample is then contacted with a test compound. The reaction heat rate is then determined in the biological process sample and compared with the reaction heat rate of the biological process in the absence of the test compound. A difference in the reaction heat rate of the biological process sample in the presence and absence of a test compound is indicative that the compound is modulating a biological process.

Examples of a biological process include any complex biochemical process. In other embodiments, the biological process is a coupled process of chemical reactions comprising a metabolic pathway including, but not limited to, bacterial cell wall biosynthesis, aromatic amino acid biosynthesis, oxidative phosphorylation, citric acid cycle, glycolysis, gluconeogenesis, photophosphorylation, lipid biosynthesis, glycosylation and cofactor biosynthesis. In other embodiments, the biological process is a coupled process of chemical reactions comprising a macromolecular process including, but not limited to, DNA replication, RNA polymerization, transcription/translation, protein synthesis, protein secretion, microtubule polymerization, histone deacetylation, proteosome protein degradation, chaperone mediated protein folding, and cellular respiration.

The biological process sample used in the methods of the invention can be prepared as known in the art. The biological process sample can be prepared from biological materials such as cells or tissues, or can be prepared by adding two or more biomolecules that are involved in the biological process of interest together with their chemical reactants. For example, to identify a modulator of eukaryotic cellular respiration one skilled in the art would prepare a mitochondrial preparation. The sample, a mitochondrial pellet, can be prepared by, for example, homogenizing liver tissue, centrifuging at low speed, e.g., 500×g, to remove large cells and tissue fragments, and then centrifuging the supernatant at a high speed, e.g., 9,000×g. Pyruvic acid oxidation coupled to ATP synthesis and modulation of this process could then be followed by monitoring the reaction heat rate in response to added exogeneous pyruvic acid in the presence and absence of a test compound. Similarly, processes occurring at endoplasmic reticulum (ER), such as protein degradation can be tested, for example, by preparing liver microsomes as known in the art. For example, microsomes can be prepared by differential centrifugation to remove fractions containing cellular debris, nucleic acids, mitochondria, etc. Misfolded and/or highly aggregated proteins destined for degradation can then be used to initiate protein degradation and the reaction heat rate is then monitored. Modulation of the process can then be followed, by comparing the heat generated, in the presence and absence of a test compound, by the process.

The biological process sample can be prepared by adding two or more biomolecules and their activators together so as to re-create a biological pathway. For example, the Mur pathway can be recreated by adding MurB, MurC and their chemical reactants, for example the MurB substrates UDP-GlcNAc-EP, NADPH, and the Murc substrates not produced by the MurB reaction, for example L-alanine and ATP. In general, a test mixture containing the biomolecules of the biological process are prepared, and the biological process is then followed by monitoring the heat output upon addition of the appropriate substrates and/or activators of the biological process. Additionally, by monitoring the heat changes in the biological process in the presence and absence of test compounds, possible modulators of the biological process can be identified.

The method of the invention can be set up as known in the art. In some embodiments, the test and/or control mixtures are added to solution reservoirs on a thermal sensitive matrix, and the test compounds and/or chemical reactants are introduced using electrosmotic or vacuum-driven flow. Alternatively, the test mixture/sample and control mixtures can be added to a section of capillary tubing, a chamber enclosed in an adiabatic environment, etc.

The methods of the invention are particularly suitable for high through-put screening. For example, the methods of the invention can include providing (i) a plurality of test mixtures containing a biomolecule and (ii) a control mixture against which the reaction heat rate in the test mixture will be compared (for example the control can be water; buffer; or a test mixture which does not include one of the components of the reaction). One or more different test compounds can be added to the plurality of test mixtures and differences in the reaction heat rates of the different test mixtures in the presence of the compound(s) compared to the reaction heat rates in the absence of the test compound(s) can be used to indicate those test compounds that modulate the activity of the biological process.

Measurement of Heat Change

A process that involves a chemical change is accompanied by energy transfer between the chemical systems and its surroundings. If the chemical reaction releases energy the event is exothermic and heat is released in the surroundings. If the reaction requires energy to occur the event is endothermic and heat is absorbed from the surroundings. The methods of the present invention measure for a difference in the reaction heat rate as a means of monitoring for changes in a reaction involving a specific biomolecule or in a biological process.

The difference in a reaction heat rate can be measured by a variety of techniques known to those of skill in the art. In some embodiments of the present invention, heat rate is measured using instruments designed for calorimetry. Such calorimeters are known to the art and include commercially available instruments such as the Microcal VP ITC and the Microcal VP DSC from Microcal (Northampton, Mass.). In general, the activity of a biological process in the presence and absence of test compounds is measured by monitoring the difference in the reaction heat rate relative to a control which does not contain the test compound. Test compounds can be introduced into the samples by various methods known to those skilled in the art, including, but not limited to, injection using one or more syringes.

In some embodiments, heat change is measured using instruments designed for differential scanning calorimetry, and the change in the reaction heat rate of a biological process is measured in a test cell over a temperature range relative to a control sample measured under the same conditions, in the presence and absence of a test compound.

In some embodiments, the test compounds and/or chemical reactants are mixed and introduced into the test cell using robotic liquid handling equipment.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Inhibition of DNA Gyrase by Novobiocin as Measured by ITC

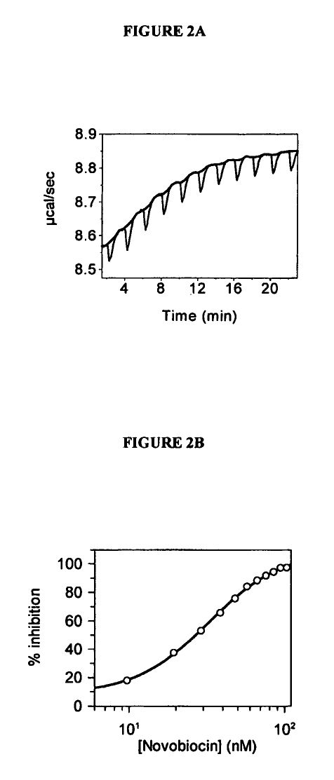

The enzyme comprising E. coli GyrA and GyrB subunits, at a concentration of 1 □M each, were incubated in 50 mM Tris (pH 7.5), 75 mM ammonium acetate, and 200 nM of 250 base pairs (bp) DNA at room temperature for 30 minutes. The enzyme stock was diluted 20-fold to a final concentration of 50 nM in assay buffer (50 mM Tris pH 7.5, 75 mM ammonium acetate, 5% glycerol, 0.5 mM EDTA, 5.5 mM magnesium chloride) that contained ATP at a final concentration of 500 μM. The diluted enzyme solution was placed in the ITC test cell and the calorimetric cells were equilibrated. Novobiocin was diluted in assay buffer at a final concentration of 2.8 μM and it was loaded into the titration syringe. The titration was started manually after the DP signal was stabilized. A total of 15 injections of 5 μl each were performed. Time spacing in between injections was set at 120 sec. Control experiments were performed under identical conditions with experiment assay buffer in the syringe. In these experiments, no decrease in DP baseline was observed after the completion of each injection peak. In separate control experiments the linearity of the reaction was checked under the same assay conditions. The reaction progress was found to be linear with time for at least 30 minutes. The inhibition curve of the ATPase activity of E. coli GyrAB by novobiocin, a potent natural-product gyrase inhibitor, was generated by using the ITC instrument (see FIGS. 2A and 2B). The reaction was started by adding ATP at a final concentration of 250 μM to a mixture of E. coli GyrAB and a 250 bp DNA. The final concentration of E. coli GyrAB in the reaction was 50 nM of each subunit and the DNA concentration was 10 nM. All measurements contained 50 mM Tris, pH 7.5, 75 mM ammonium acetate, 5% glycerol, 0.5 mM EDTA, and 5.5 mM magnesium chloride. Novobiocin was dissolved in the same buffer at a final concentration of 2.8 μM. The reaction mixture was placed in the test cell. The control cell was filled with H₂O and the test and control cells were equilibrated. Novobiocin was added in the titration syringe. Fifteen 5 μl injections were performed.

Upon novobiocin addition, the power baseline decreased after each injection peak (solid black line in FIG. 2A), an observation consistent with inhibition of enzyme activity. After a certain injection, the power baseline plateaus, an indication that all enzyme activity has been inhibited. The data were analyzed as follows: the power value (μcal/sec) after each injection is subtracted from the initial power value (before any injection occurs). Each point is then calculated as % inhibition assuming that 100% inhibition occurs where the power baseline plateaus after the n^{th} injection of inhibitor. After data analysis, an IC₅₀ value of 26 nM was obtained for novobiocin inhibition. This value is in close agreement with the value obtained by using an independent assay of enzyme activity using phosphate detection. (IC₅₀=28 nM, see Table 1).

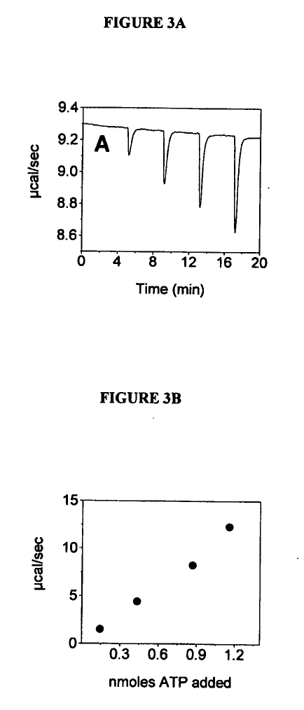

It is also possible to convert the enzyme activity expressed as power units to activity expressed as turnover number units by using a calibration curve that converts the reaction heat rate to the reaction product formation rate and calculate the % inhibition as described above. An example of such conversion is shown in FIGS. 3A and 3B.

The experiment shown in FIG. 3A was run as follows: reconstituted E. coli GyrAB (a mixture of 100 nM of each GyrA and GyrB subunits in the presence of 20 nM of a 250 base pair DNA) was added into the test cell. ATP was dissolved in the same buffer at a final concentration of 145 μM and added to the titration syringe. Four injections (1, 3, 5, and 8 μl) were completed. The injection spacings were adjusted so the power baseline returned to the initial position between injections, an indication that the reaction was completed for each injection of substrate (see FIG. 3A). The area under each peak represents the total heat released for the conversion of the added ATP to ADP and Pᵢ. By plotting the concentration of ATP added in each injection vs. area of the injection peak it is possible to obtain a calibration curve the slope of which is the conversion factor of heat released perproduct formed) in units of μcal/nmole. The slope of the curve shown in FIG. 3B was calculated to be 11 kcal/mole. This number is consistent with the number of 13 kcal/mole reported for the energy released in ATP hydrolysis. Measuring kinetic parameters such as K_{M, ATP}, k_{cat} and K_{i, ADP} by ITC contributed to further characterization of E. coli GyrAB. These values are compared with those obtained by an independent assay of enzyme activity using phosphate detection (Table 1).

Table 1 shows kinetic parameters of the E. coli GyrAB ATPase reaction obtained by ITC. For comparison, values obtained by an independent method of phosphate detection are included.

TABLE 1

| | K_{M. ATP} | K_{i, ADP} | Novobiocin IC₅₀ |
|---|---|---|---|
| Method | (μM) | (μM) | (μM) |
| ITC | 166 | 18 | 0.026 |
| Phosphate detection | 145 | 18 | 0.028 |

### Example 2

### MurC

E. coli MurC was diluted in assay buffer (50 mM Tris pH 8.0, 1 mM Tris (2-carboxyethyl) phosphine hydrochloride (TCEP), 0.01% (w/v) Triton X-100, 20 mM ammonium formate) to a final concentration of 10 nM. The buffer also contained the MurC substrates at the following concentrations in the ITC test cell: 100 μM UDP-MurNAc, 275 μM L-alanine, and 300 μM ATP. β,γ-Methyleneadenosine 5′-triphosphate (AMP-PCP) was dissolved in assay buffer at a final concentration of 2.8 mM and was loaded into the titration syringe. The titration was started manually after the DP signal was stabilized. A total of 16 injections (2, 2, 4, 4, 4, 4, 8, 8, 16, 16, 32, 32, 32, 32, 32, and 32 μl) were performed. Time spacing in between injections was set at 120 sec for all injections.

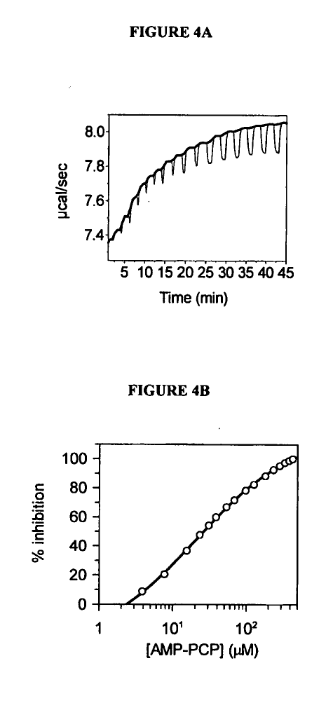

The inhibition of MurC by AMP-PCP is shown in FIG. 4. All the reactions were carried in 50 mM Tris, pH 8.0, 1 mM TCEP, 0.01% (w/v) Triton, and 20 mM ammonium formate. The final concentration of E. coli MurC was 10 nM. The final concentrations of the MurC substrates were as follows: 100 μM UDP-MurNAc, 275 μM L-alanine, and 300 μM ATP. The reaction was initiated with the addition of MurC and placed into the test cell. AMP-PCP was dissolved in the buffer described above at a final concentration of 2.8 mM and placed into the titration syringe.

The raw ITC data are shown in FIG. 4A. An IC₅₀ value of 27 μM was obtained for MurC inhibition by AMP-PCP (see FIG. 4B). This value compared well with the value of 40 μM obtained using an independent assay of enzyme activity by detecting inorganic phosphate formation.

Other kinetic measurements were performed to further characterize E. coli MurC by ITC. Table 2 shows kinetic parameters of the E. coli MurC reaction obtained by ITC. For comparison, values obtained by an independent method of phosphate detection are included.

TABLE 2

| | | | | | AMP-PCP |
|---|---|---|---|---|---|
| | K_{M, ATP} | K_{M, UDP-MurNAc} | K_{M, ALA} | k_{cat} | IC₅₀ |
| Method | (μM) | (μM) | (μM) | (min⁻¹) | (μM) |
| ITC | 61 | 21 | 44 | 50 | 27 |
| Phosphate | 101 | 19 | 56 | 71 | 35 |
| detection | | | | | |

### Example 3

### MurB

E. coli MurB was diluted in assay buffer (50 mM Tris pH 8.0, 1 mM TCEP, 20 mM KCl, 0.005% Triton X-100) to a final concentration of 1.3 nM. The buffer contained the MurB substrates at the following concentrations in the ITC test cell: 100 μM NADPH and 60 μM UDP-GlcNAc-EP. DMSO was also added at a final concentration of 2.7% (v/v). The stock compound ((2R)-2-{2-[3-(4-tert-butylphenoxy)phenyl]-4-oxo-1,3-thiazolidin-3-yl} hexanoic acid was prepared in DMSO and diluted in assay buffer to a final concentration of 2.8 mM. The titration was started manually after the DP signal stabilized and a total of 10 injections (8, 8, 17, 17, 24, 24, 32, 32, and 50 μl) were performed. Time spacing in between injections was set at 100, 100, 120, 120, 120, 150, 150, 150, 150, and 180 sec respectively.

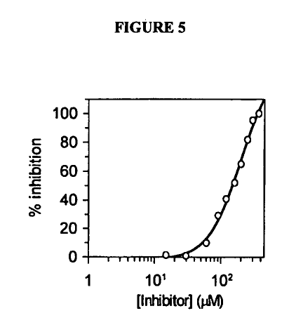

The inhibition of E. coli MurB by (2R)-2-{2-[3-(4-tert-butylphenoxy)phenyl]-4-oxo-1,3-thiazolidin-3-yl} hexanoic acid, a 4-thiazolidinone compound, was studied by the same method. Reactions were carried in 50 mM Tris, pH 8.0, 1 mM TCEP, 20 mM KCl, 0.005% Triton, in the presence of 100 μM NADPH and 60 μM EP. The concentration of MurB was 1.3 nM. An IC₅₀ value of 154 μM was obtained by the method described above (see FIG. 5). An IC₅₀ value of 101 μM was obtained by an absorbance method which measured MurB activity by measuring NADPH depletion at 340 nm.

Other kinetic measurements were performed to further characterize E. coli MurB by ITC. Table 3 shows kinetic parameters of the E. coli MurB reaction obtained by ITC. For comparison, values obtained by an independent method of NADPH depletion detection are included.

TABLE 3

| | | | 4-thiazolidinone |
|---|---|---|---|
| | K_{M, NADPH} | K_{M. UDP-GlcNAc}_{^{−}}_{EP} | analogue IC₅₀ |
| Method | (μM) | (μM) | (μM) |
| ITC | 9 | 8 | 154 |
| Absorbance | 9 | 6 | 101 |

### Example 4

### Inhibition of the Transcription/Translation Process by Tetracycline as Measured by ITC

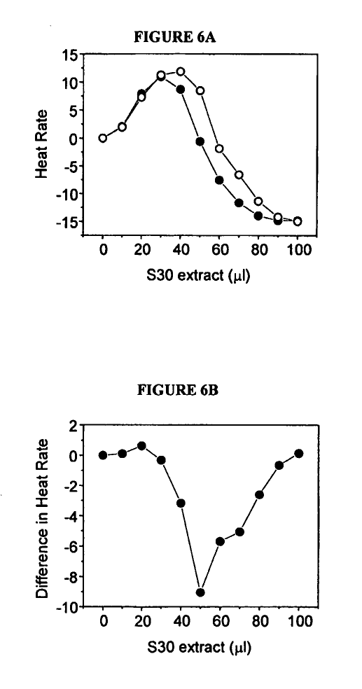

The inhibition of the transcription/translation process by tetracycline is shown in FIGS. 6A and 6B.

A reaction mix was prepared, containing 17.5 mM Tris acetate, 95.2 mM potassium acetate, 15 mM ammonium acetate, 5 mM TCEP, 5 mM ATP, 1.25 mM each of four nucleotide triphospates, (adenosine triphosphate (ATP), cytidine triphosphate (CTP), guanidine triphosphate (GTP), and uridine triphosphate (UTP)), 87 mM phospho(enol)pyruvate (PEP), 0.25 mg/ml transfer ribonucleic acid (tRNA), 350 mg polyethyleneglycol (PEG), 0.05 mg/ml folinic acid, 2.5 mM cyclic adenosine monophosphate (cAMP), 2 mM isopropyl β-D-1-thiogalactopyranoside (IPTG), and 18.9 mM each of the 20 amino acids.

A 2 ml substrate mixture was prepared, containing 800 μl of the reaction mixture, 15 μl of 1 mg/ml pBEST luc™ vector DNA (Promega) and 1185 μl H₂O.

The same substrate mixture was prepared, containing 800 μL of the reaction mixture, 15 μl of 1 mg/ml pBEST luc™ vector DNA (Promega), 20 μl of 20 mM tetracycline in dimethyl sulfoxide (DMSO), and 1165 μl H₂O.

S30 extract (containing ribosomes and a wide variety of enzymes and cofactors needed for the transcription/translation process) from bacterial lysate was prepared and added in the titration syringe. S30 extract was titrated in the test cell containing the substrate mixture. 10 injections, 10 μl each, were initially performed upon S30 extract addition. The power baseline increased after each injection (indicative of an endothermic event). At the fifth injection of S30 extract the power baseline decreased and continued decreasing after subsequent injections (indicative of exothermic event) (see FIG. 6A, closed circles).

S30 extract was titrated in the test cell containing the substrate mixture and tetracycline (see FIG. 6A, open circles). Initially the same heat rate changes were observed, but at the fifth injection of S30 extract (exothermic event starts to take place), differences in the magnitude of the heat rate change were evident. These differences were calculated and plotted as differences in the heat change observed upon titrating S30 extract in the substrate mixture in the presence or absence of tetracycline (see FIG. 6B). The difference in heat rate is defined as the heat rate observed in the presence of tetracycline minus the heat rate observed in the absence of tetracycline. It is evident from FIG. 6B that a portion of the transcription/translation process is inhibited by tetracycline since at the fifth injection of S30 extract the heat change is smaller in the presence of tetracycline (negative heat rate difference)) than that in the absence of tetracycline. The effect of tetracycline in the heat rate of the process becomes less evident upon continuing S30 injections (the differences in the heat rate observed in the presence and absence of tetracycline become smaller).

The foregoing examples are meant to illustrate the invention and are not to be construed to limit the invention in any way. Those skilled in the art will recognize modifications that are within the spirit and scope of the invention.

We claim:

1. A screening method for identifying a modulator of a biological process, the method comprising:
providing a test mixture from a biological process under conditions that support biological activity;
adding a test compound to the test mixture; and
detecting a difference in the reaction heat rate in the presence of the compound compared to the reaction heat rate in the absence of the test compound, wherein the difference is indicative that the test compound modulates the activity of the biological process.

2. The method of claim 1 wherein the biological process is transcription or translation.

3. The method of claim 1 wherein the biological process is selected from the group consisting of bacterial cell wall biosynthesis, DNA replication, protein degradation, and protein secretion.

4. A screening method for identifying a modulator of a biological process, the method comprising:
providing a test mixture from a biological process and a control;
adding a test compound to the test mixture;
equilibrating heat between the test mixture and the control;
initiating a reaction in the test mixture and the control; and
detecting a difference in the reaction heat rate between the test mixture and control, wherein the difference is indicative that the test compound modulates the activity of the biological process.

5. The method of claim 4 wherein the biological process is transcription or translation.

6. The method of claim 4 wherein the biological process is selected from the group consisting of bacterial cell wall biosynthesis, DNA replication, protein degradation, and protein secretion.

7. A screening method for identifying a modulator of a biological process, the method comprising:
providing a test mixture from a biological process under conditions that promote activity and a control;
equilibrating heat between the test mixture and the control;
adding a test compound to the test mixture; and
detecting a difference in the reaction heat rate in the presence of the compound compared to the reaction heat rate in the absence of the test compound, wherein the difference is indicative that the test compound modulates the activity of the biological process.

8. The method of claim 7 wherein the biological process is transcription or translation.

9. The method of claim 7 wherein the biological process is selected from the group consisting of bacterial cell wall biosynthesis, DNA replication, protein degradation, and protein secretion.

10. A screening method for identifying a test compound that modulates the activity of a biomolecule, the method comprising:
providing a test mixture comprising a biomolecule under conditions that support the activity of the biomolecule;
adding a test compound to the test mixture; and
detecting a difference in the reaction heat rate in the presence of the compound compared to the reaction heat rate in the absence of the test compound, wherein the difference is indicative that the test compound modulates the activity of the biomolecule.

11. The method of claim 10 wherein the biomolecule is selected from the group consisting of a protein, an oligonucleotide, a DNA or RNA polynucleotide, carbohydrate, and a lipid.

12. The method of claim 1 wherein the protein is an enzyme or a polypeptide.

13. The method of claim 12 wherein the enzyme is from a prokaryote, a eukaryote, a virus or a fungus.

14. The method of claim 12 wherein the enzyme is a topoisomerase.

15. The method of claim 12 wherein the enzyme is from a bacterium.

16. The method of claim 15 wherein the enzyme is DNA gyrase and topoisomerase IV.

17. The method of claim 12 wherein the enzyme is selected from the group consisting of oxidases/reductases, kinases, ligases, and phosphatases.

18. The method of claim 12 wherein the enzyme is MurB, MurC or DNA ligase.

19. The method of claim 12, wherein the enzyme is involved in cell wall biosynthesis and transmembrane signaling, translation, transcription, replication, protein secretion, or cofactor biosynthesis.

20. A screening method for identifying a compound that modulates the activity of a biomolecule, the method comprising:
providing a test mixture comprising a biomolecule and a control;
adding a test compound to the test mixture;
equilibrating heat between the test mixture and the control;
initiating a reaction in the test mixture; and
detecting a difference in the reaction heat rate between the test mixture and control, wherein the difference is indicative that the test compound modulates the activity of the biomolecule.

21. The method of claim 20 wherein the biomolecule is selected from the group consisting of a protein, an oligonucleotide, a DNA or RNA polynucleotide, or a lipid.

22. The method of claim 21 wherein the protein is an enzyme or a polypeptide.

23. A screening method for identifying a compound that modulates the activity of a biomolecule, the method comprising:
providing (i) a test mixture comprising a biomolecule under conditions that promote the activity of the biomolecule and (ii) a control;
equilibrating heat between the test mixture and the control;
adding a test compound to the test mixture; and
detecting a difference in the reaction heat rate in the presence of the compound compared to the reaction heat rate in the absence of the test compound, wherein the difference is indicative that the test compound modulates the activity of the biomolecule.

24. The method of claim 23 wherein the biomolecule is selected from the group consisting of a protein, an oligonucleotide, a DNA or RNA polynucleotide, or a lipid.

25. The method of claim 24 wherein the protein is an enzyme or a polypeptide.
